(19) United States
(12) Patent Application Publication
(10) Pub. No.: US 2002/0197417 A1
(43) Pub. Date: Dec. 26, 2002

(54) Method for producing DNA chip, and DNA chip obtained thereby

(75) Inventors: Eiichi Nakamura, Tokyo (JP); Kenichi Shibayama, Tokyo (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: KISHIMOTO SANGYO CO., LTD., Osaka-shi (JP)

(21) Appl. No.: 10/156,020

(22) Filed: May 29, 2002

(30) Foreign Application Priority Data
May 29, 2001 (JP) 2001-160935

(51) Int. Cl. C23C 8/00; C23C 8/54; C12M 1/00

(52) U.S. Cl. 427/585; 427/590; 435/283.1

(57) ABSTRACT

This invention attempts to provide a method for producing a DNA chip which can be accomplished in simple steps at a low cost, and wherein use of the resulting DNA chip reduces loss of probes and sample substances in the washing step enabling efficient use of such probe and sample. This invention also attempts to provide a DNA chip produced by such method. Accordingly, a method for producing a DNA chip comprising a substrate and a DNA-binding layer formed on the substrate wherein said DNA-binding layer is a diamond like film having a DNA-binding group is provided, and this method comprises the steps of: reducing pressure of a vacuum chamber to a predetermined degree of vacuum; feeding the chamber with a gas which is the source of said diamond like film; feeding the chamber with a gas which is the source of nitrogen; and forming the diamond like film having a DNA-binding group on the substrate by CVD. Also provided is the DNA chip produced by such method.

### BACKGROUND OF THE INVENTION

1. Technical Field

This invention relates to a DNA chip which is useful in assaying gene expression, gene mutation, gene polymorphism, and the like.

2. Background Technology

Expression of genes in cells and tissues has been assayed by Northern blotting (or dot blotting) wherein RNA from various cells or tissues is immobilized on a membrane, and the RNA is hybridized by using a probe specific to the analyte gene; RT-PCR using a primer specific to the analyte gene; and the like.

There is, however, an increasing demand for an assay wherein a large number of genes can be assayed at once, as a result of progress in gene researches and the associated increase in the number of analyte genes as well as progress of the Genome Project and the application of its outcome in the field of medicine.

In view of such demands, various techniques have been developed that utilize microarray, DNA chip, or the like. Such techniques share the common feature that several thousands DNA fragments of different type are immobilized on a glass substrate (which is referred to as a DNA chip or a bio chip), and the target DNA fragment is detected at a high sensitivity by means of the hybirdization between the immobilized DNA fragment and the very minute amount of the labeled target DNA fragment.

Such techniques have enabled to assay a large number of genes of human and other mammals or even the entire genes of a microorganism including several thousand genes on several DNA chips. Also enabled are assays of the amount gene expression for the entire genes by using labeled RNAs. Assay of gene deletion and other mutation has also been enabled by labeling the genomic DNA.

When a DNA chip is produced by a procedure other than the “on chip” synthesis (i.e. the procedure wherein the DNA fragments to be immobilized on the surface of the substrate are directly synthesized on the surface of the substrate), the DNA fragments that had been produced beforehand have to be spotted on the surface of the substrate, and immobilized by utilizing electrostatic interactions or covalent bonding.

FIG. 2 is a view explaining the principle of this procedure. FIG. 2, step A shows a microplate 22 with probe DNAs 21 of different type placed in the microplate 22. In the meanwhile, a glass plate as shown in FIG. 2, step B is prepared for uses as a plate 23, and as shown FIG. 2, step C, the surface of the plate 23 is coated with a binder 24 such as poly-l-Lysine which binds the DNA to the glass. Next, the probe DNA 21 in the microplate 22 is attached to a pin, and the DNA 21 attached on the pin is brought in contact with the glass plate 23 that had been coated with the binder (poly-l-Lysine) 24 for the DNA and the glass in order to spot the DNA 1 on the coated glass. This procedure is repeated until the spotting of all probe DNAs in the microplate 22 has been completed, and a DNA chip shown in FIG. 2, step D is thereby produced. As described above, DNA chips have been produced by preliminarily coating the entire surface of a plate with a binder of the DNA and the glass, and thereafter spotting the DNA on the plate coated with the binder.

The hybridization of the DNA chip is accomplished by placing the DNA chip wherein the probe DNAs had been spotted and immobilized on the glass plate by means of the binder and the sample DNA that had been labeled with a fluorescent substance in a hybridization solution in order to promote the hybridization. The hybridization solution is a mixed solution of formaldehyde, SSC (NaCl, trisodiumcitrate), SDS (sodium dodecyl sulfate), EDTA (ethylenediamidetetraacetic acid), distilled water, and the like, and mixing ratio may vary depending on the characteristic of the DNA used.

In this step, the sample DNA and the probe DNA on the DNA chip will bind to each other by forming a double helix structure if these DNAs have complementary DNA strands. On the other hands, the DNAs will not bind to each other if the DNAs are not complimentary to each other, and the sample DNA that had been labeled with a fluorescent substance either remains in the hybridization solution or becomes bound to the binder coated on the glass plate to remain as a garbage.

When the glass plate is washed in a water tank or the like to thereby remove the sample DNA that had been labeled with a fluorescent substance remaining on the glass plate, the sample DNA that had failed to bind to the probe DNA is washed away. The hybridization is then detected by exciting the fluorescent label on the sample DNA that is bonded to the probe DNA by the light energy emitted from the predetermined light source, and scanning the light emitted by the excitation of the fluorescent label using a photosensor such as CCD.

However, the binder such as poly-l-Lysine used to bind the DNA and the glass is insufficient in the binding strength with the DNA, and the probe DNA often became detached from the substrate together with the hybridized sample in the step of washing by water. The loss of the probe DNA and the sample DNA due to such insufficient binding often reached as high as 70%, and it has been the state of art that the expensive probe DNA and the precious sample DNA are being wasted.

In order to obviate such problem, various materials have been examined for use as a binder. For example, DLC (diamond like carbon) film is a promising material which has excellent heat resistance and durability. However, in the case of DLC, the surface of the film formed had to be chlorinated and then aminated by substituting the chlorine with ammonia gas.

Such surface treatments which resulted in the complicated production process and poor yield of the resulting product had been a stumping block in reducing the production cost. In view of the increasing clinical applications of the DNA chips, it is particularly important to provide a DNA chip at a price affordable for mass public in order to improve both public health and medical technology.

### SUMMARY OF THE INVENTION

An object of the present invention is to provide a method for producing a DNA chip which can be accomplished in simple steps at a low cost, and wherein use of the resulting DNA chip reduces loss of probes and sample substances in the washing step to enable efficient use of such probe and sample. Another object of the invention is to provide the DNA chip produced by such method.

The object as described above is achieved by the present invention which is constituted as described below.

(1) A method for producing a DNA chip comprising a substrate and a DNA-binding layer formed on the substrate wherein said DNA-binding layer is a diamond like film having a DNA-binding group; comprising the steps of

reducing pressure of a vacuum chamber to a predetermined degree of vacuum;

feeding the chamber with a gas which is the source of said diamond like film;

feeding the chamber with a gas which is the source of nitrogen; and

forming the diamond like film having a DNA-binding group on the substrate by CVD.

(2) The method for producing a DNA chip according to the above (1) wherein said diamond like film is a diamond like nanocomposite (DLN) film comprising at least carbon, silicon, oxygen, and hydrogen.

(3) The method for producing a DNA chip according to the above (1) wherein said diamond like film is a diamond like carbon (DLC) film comprising at least carbon and hydrogen.

(4) The method for producing a DNA chip according to the above (1) or (2) wherein the source gas for said diamond like film is the one obtained by heating a silicone oil.

(5) The method for producing a DNA chip according to the above (4) wherein said nitrogen source is included in the silicone oil.

(6) A DNA chip which is produced by the method of claims 1 to 5, and which has a DNA-binding layer having a DNA-binding group.

### BRIEF DESCRIPTION OF THE DRAWINGS

FIG. 1 is a view schematically illustrating the apparatus used in producing the DNA chip of the present invention. FIG. 2 is a view schematically illustrating the production process of the DNA chip.

### PREFERRED EMBODIMENTS OF THE INVENTION

In the method of the present invention for producing a DNA chip, the DNA chip comprising a substrate and a layer containing a DNA-binding group is formed by directly depositing an amino group-modified diamond like film on the substrate so that the diamond like film functions as the layer containing a DNA-binding group.

To be more specific, the method of the present invention comprises the steps of reducing the pressure of a vacuum chamber to a predetermined degree of vacuum, feeding the chamber with a gas which is the source of the diamond like film, feeding the chamber with a gas which is the source of nitrogen, and forming the diamond like film having a DNA-binding group on the substrate by CVD so that the diamond like film may serve a DNA-binding film. It is also acceptable that the nitrogen source is included in the source gas for the diamond like film.

The term “diamond like film” used herein designates a film of diamond like carbon (DLC) or diamond like nanocomposite (DLN).

A diamond-like carbon (DLC) film is a carbon film of high hardness obtained by exciting and decomposing a hydrocarbon. The DLC film is also referred to as an i-carbon film. With respect to the DLC film, reference is made to, for example, JP-A 62-145646, JP-A 62-145647, and New Diamond Forum, Vol. 4, No. 4 (Oct. 25, 1988).

As described in New Diamond Forum, DLC exhibits a broad absorption peak around 1550 cm⁻¹ (1520 to 1560 cm⁻¹) as analyzed by Raman spectroscopy and thus has a distinct structure distinguishable from diamond exhibiting a sharp peak at 1333 cm⁻¹ and graphite exhibiting a sharp peak at 1581 cm⁻¹.

The DLN (Diamond Like Nanocomposite) is an amorphous, hard, thin film which has a double structure wherein mutually independent carbon network structure and silicon network structure interpenetrate with each other, and the DLN may be represented as

a(CHₓ)_{y}·a(SiO_{z})_{w}.

Also, the DLN may further comprise a metal element as the a third component. The DLN is described, for example, in V. F. Dorfman and B. N. Pypkin, Surface and Coating Technology 48, 193(1991), and U.S. Pat. No. 5,352,492. The structure of the DLN may be confirmed by STM, AFM, X ray analysis, electron beam analysis, TEM, or the like. For example, peaks indicating the presence of Si—O bond are found in FTIR spectrum at 1010 cm⁻¹ and 800 cm⁻¹.

In a typical process wherein the DLC or the DLN is used for the binder, a DLC or a DLN film is formed by means of CVD or the like, and after washing the resulting film, carbon atoms on the surface the DLC or the DLN film are substituted with chlorine by UV irradiation. Next, the chlorine is substituted in the presence of ammonia gas by UV irradiation to thereby accomplish amination of the surface carbon atoms and facilitate the covalent bonding. The amino group on the surface of the DLC or the DLN is then allowed to undergo amide bonding with the DNA fragment.

As described above, the conventional DNA chip produced by such procedure suffers from insufficient production efficiency, low yield, and undesirable wasting of the raw materials. In contrast, the DNA chip produced by the method of the present invention has a thin, uniform binder-containing layer free from pin holes, which allows bonding and immobilization of the DNA fragments in favorable state. The production step of the present invention is also simple since the amino group required for the amide bonding is introduced simultaneously with the formation of the DLC or the DLN film.

In particular, the DNA chip produced by using the DLN film enjoys excellent adhesion to the substrate, high stability, satisfactory heat resistance, photoresistance, and mechanical strength, and accordingly, fully endures repeated use. Efficient use of the DNA chip is thereby enabled.

In producing the diamond like film, the source material is supplied as a source gas. A source gas capable of providing carbon and hydrogen is supplied for producing a DLC film, and a source gas capable of providing carbon, silicon, oxygen, and hydrogen is supplied for producing a DLN film.

Exemplary carbon sources include CO, CO₂, CH₃, and C₂H₅, and exemplary silicon sources include silane and methylsilane. Exemplary oxygen sources include O₂, and exemplary hydrogen sources include H₂. These materials may be supplied as a mixture of source gases, or as a single gas source supplying a plurality of materials.

These source gases may be supplied in an adequate necessary amount depending on the material used and the type of the film formed.

Use of a silicone oil is particularly preferable when a DLN film is formed in the present invention. The silicone oil which may be used in forming the DLN film is an organosiloxane compound which simultaneously serve silicon source, carbon source, oxygen source, and hydrogen source for the DLN, and elements constituting the DLN can be supplied at a good balance by using one single material. The gaseous silicone oil used can be obtained by heating and vaporizing the silicone oil.

Exemplary silicone oils include dimethylsilicone, dialkylpolysiloxiane, dialkoxypolysiloxiane, phenylpolysiloxiane, fluoroalkylpolysiloxiane, and amino-modified silicone oils. Use of an unmodified silicone oil such as dimethylsilicone is preferred, and use of dimethylsilicone is particularly preferable. The silicone oil is available, for example, from Shinetsu Chemical under the product names of KF96 and KF69.

The source silicone oil is held in a predetermined container and placed in the vacuum chamber. The container used for the silicone oil is not particularly limited as long as the container has low reactivity with the silicone oil and the container withstands certain degree of temperature to which it is heated. Exemplary containers include containers such as an evaporation boat and a crucible comprising a material such as platinum or PBN which are typically used in the vacuum evaporation.

The source silicone oil is heated to a temperature which allows vaporization of the silicone oil in the vacuum chamber, and most typically to a temperature in the range of about 200 to 600° C., and preferably to the range of about 300 to 400° C.

The vacuum chamber is preferably evacuated to a degree of vacuum of 10⁻³ Torr (1.33×10⁻¹ Pa) or less, and more preferably, to 10⁻⁴ Torr (1.33×10⁻² Pa) or less.

Typical examples of the source gas which may be used in incorporating amino group in the DLC or the DLN include N₂ and NH₃, and such source gas may be introduced in the vacuum chamber at a flow rate of about 1 to 100 SCCM, and in particular, at about 1 to 50 SCCM. Such source gas may be introduce in combination with H₂, CH₄, and the like.

Introduction of such source gas, however, is not required when the silicone oil used contains a nitrogen source. Exemplary such nitrogen source-containing silicone oil include amino-modified silicone oils.

The DLC or the DLN film is formed by introducing the source materials in the vacuum chamber and conducting the chemical vapor deposition (CVD). The CVD is prefereably accomplished by bias-assisted plasma CVD wherein self bias or other bias is applied. Use of an RF power source is preferable in the CVD, and the RF power is preferably about 50 w to 2 kw. The bias voltage is typically −50 V to −5 kV, the total pressure is 0.02 to 0.2 Torr (2.66 to 26.6 Pa), the reaction time is 10 to 120 minutes, the distance between the electrodes is, for example, about 4 cm, the total gas flow rate is 0.2 to 100 SCCM, and the substrate temperature is 10 to 300° C.

It is to be noted that a glass substrate is placed on the substrate of the vacuum chamber so that the DLC film or the DLN film can be formed on the glass substrate. A counter electrode is arranged above the substrate at a predetermined distance.

The resulting amino-modified DLC or DLN film (DNA binder-containing layer) is preferably washed with distilled water before further processing.

The DNA chip having a DNA binder-containing layer produced by the present invention exhibits good binding to the probe DNA, and detachment of the DNA in the step of washing with water is prevented to enable efficient use of the DNA material. This DNA chip also exhibits excellent heat resistance, weatherability, and mechanical strength, and repeated use of the chip is thereby enabled.

Prior to the immobilization of the probe on the chip, the amino group as described above is preferably chemically modified by carboxylation using an acid chloride.

The probe is immobilized by amide bonding of the single strand oligonucleotide (hereinafter referred to as oligonucleotide A) to the chemically modified moiety as described above. The chemically modified hydrocarbon group terminal is preferably activated to facilitate the probe immobilization, and use of carbodiimide for the dehydrating/condensing agent is particularly preferable.

It is to be noted that the chip may be prepared by leaving the primary amino group formed by amination (with no further carboxylation) on the surface of the chip, and reacting the amino group with one ester group of an activated diester having ester groups such as N-hydroxysuccinimide or p-nitrophenol for dehydration/condension.

The substrate preferably comprises a transparent glass, silicone, polyethylene terephthalate, cellulose acetate, bisphanol A polycarbonate or other polycarbonate, polystylene, polymethyl methacrylate, or other polymer. Among these, use of a glass or silicone is preferable in view of the ease of surface treatment and ease of analysis using a fluorescent scan system. Use of a glass plate having a silica surface layer is also preferable. The substrate may preferably have a thickness in the range of 100 to 2000 μm.

Two types of DNA fragments may be used for the probe depending on the purpose of the assay. In assaying gene expression, use of a polynucleotide such as cDNA, a part of the cDNA, or EST is preferable. Such polynucleotide may have unknown function. However, the polynucleotide is typically prepared by means of PCR using a cDNA library, a genomic library, or the entire genome for the template on the basis of the sequence registered in the data base (hereinafter referred to as “PCR product”). The polynucleotide may be the one which has not been amplified by PCR. In order to assay mutation or polymorphism of the gene, various oligonucleotides corresponding to the mutation or the polymorphism are preferably synthesized on the basis of the referential known sequence. In assaying the nucleotide sequence, 4n (n: length of the nucleotide) types of the oligonucleotides are preferably synthesized for use. The DNA fragment may preferably have a known nucleotide sequence.

The poly/oligo nucleotide may have any desired sequence. However, the sequence of the poly/oligo nucleotide at the end (3′ terminal) which is not to be immbobilized should be designed such that the double strand poly/oligo nucleotide produced by the subsequent hybridization with another poly/oligonucleotide includes a cleavage site for a restriction enzyme. In view of facilitating the amide bonding with the chemically modified chip, it is also preferable that the end of the poly/oligo nucleotide to be immobilzed on the chip includes 1 to 10 nucleotides having a primary amine such as adenine, cytosine, or guanine.

The spotting of the DNA fragments is preferably accomplished by dispensing the aqueous solutions or suspensions of the DNA fragments in an aqueous medium on a 96 well or 384 well plastic plate, and dropping the dispensed aqueous solution onto the substrate by using a spotter.

The number of the DNA fragments spotted is preferably in the range of 10² to 10⁵ types/cm² of the substrate surface. The amount of the DNA fragments is preferably in the range of 1 to 10 moles and up to several ng in weight. As a result of such spotting, the aqueous solutions of the DNA fragment will be immobilized on the surface of the substrate in the form of dots which are arranged at an interval of 0 to 1.5 mm, and most preferably 100 to 300 μm. The size of one dot is preferably such that the diameter is in the range of 50 to 300 μm. The amount of the DNA fragment spotted is preferably in the range of 100 pL to 1 μL, and most preferably in the range of 1 to 100 nL.

After the spotting, the chip may be optionally incubated as desired. The chip is then washed for the removal of the DNA fragment which failed to be immobilized.

The dots which are formed on the surface of the substrate as described above are substantially round in shape. Consistency of the dot shape is particularly important in the case of quantitatively analyzing the gene expression or one base mutation.

The life of the thus produced DNA chips are considerably long. In the case of a cDNA chip having cDNAs immobilized thereon, the chip has a life of several weeks while the chip may have an even longer life in the case of an oligodeoxynucleotide chip wherein oligodeoxynucleotides are immobilized. Such DNA chip is utilized in the monitoring of gene expression, determination of the nucleotide sequence, assay of mutation, assay of polymorphism, and the like. The principle of the detection is hybridization of the immobilized probe with the labeled target nucleic acid.

Preferably, the target nucleic acid used for the sample is a sample of DNA fragment or RNA fragment having an unknown sequence and unknown function.

In the case of assaying the gene expression, the target nucleic acid is preferably the one isolated from an eukaryotic cell or a tissue sample. When the target is the genome, the target nucleic acid is preferably the one isolated from a tissue sample other than erythrocyte. The tissue other than erythrocyte may preferably be peripheral blood lymphocyte, skin, hair, sperm, or the like. When the target is mRNA, the sample is preferably extracted from a tissue sample wherein the mRNA is expressed. The mRNA is preferably made into a labeled cDNA by incorporating the labeled dNTP (“dNTP” designates a deoxyribonucleotide wherein the nucleotide is adenine (A), cytosine (C), guanine (G), or thymine (T)) by means of reverse transcription. The dNTP used is preferably dCTP in view of the chemical stability. The amount of the mRNA required for one hybridization is preferably up to several μg although such amount may differ according to the liquid amount and the labeling method. It is to be noted that, when the DNA fragments on the DNA chip are oligodeoxynucleotides, molecular weight of the target nucleic acid is preferably reduced before the assay. In the case of a prokaryotic cell, labeling of the entire RNA is preferable in view of the difficulty of the selective extraction of the mRNA.

In order to assay the mutation or the polymorphism, the target nucleic acid is preferably prepared by conducting PCR of the target region in the reaction system containing the labeled primer or the labeled dNTP.

The labeling method used may be the one using RI or the one not using the RI among which the latter non-RI methods being the preferred. Exemplary non-RI method include fluorescent labeling, biotin labeling, and chemiluminescent labeling methods, and use of a fluorescent labeling method is preferred. Any fluorescent substance may be used as long as the substance is capable of bonding to the base moiety of the nucleic acid. However, use of a cyanine dye (for example, Cy3 or Cy5 of Cy Dye TM series), rhodamine 6G reagent, N-acetoxy-N₂-acetylaminofluorene (AAF), or AAIF (iodine derivative of AAF) is preferred.

The hybridization is preferably accomplished by preparing an aqueous solution having the labeled target nucleic acid dissolved or dispersed therein, dispensing the aqueous solution on a 96 well or 384 well plastic plate, and spotting the aqueous solution onto the DNA chip that had been produced as described above. The amount spotted is preferably in the range of 1 to 100 nL. The hybridization is preferably conducted at a temperature in the range of room temperature to 70° C. and for a period of 6 to 20 hours. After completing the hybridization, the DNA chip is preferably washed with a mixture of a surfactant and a buffer solution for removing the target nucleic acid which failed to hybridize. Exemplary surfactants include sodium dodecylsulfate (SDS). Exemplary buffer solutions include citrate buffer solution, phosphate buffer solution, borate buffer solution, Tris buffer solution, and Good's buffer solution, and use of citrate buffer solution is preferred.

The feature characteristic to the hybridization using a DNA chip is the drastically reduced amount of the labeled nucleic acid. Therefore, careful selection of the optimal conditions for the hybridization is required in accordance with the length of the DNA fragment immobilized on the substrate and the type of the labeled target nucleic acid. In the case of assaying gene expression, the hybridization is preferably conducted at low stringency for a long time to thereby enable detection of a gene which has been expressed at a low level. In the case of assaying one base mutation, the hybridization is preferably conducted at a high stringency for a short time. In the hybridization using a DNA chip, two types of target nucleic acids each labeled with different fluorescent substance may be also used on the same one DNA chip to thereby enable comparison or quantitative evaluation of the expressed amount.

### EXAMPLES

### Example 1

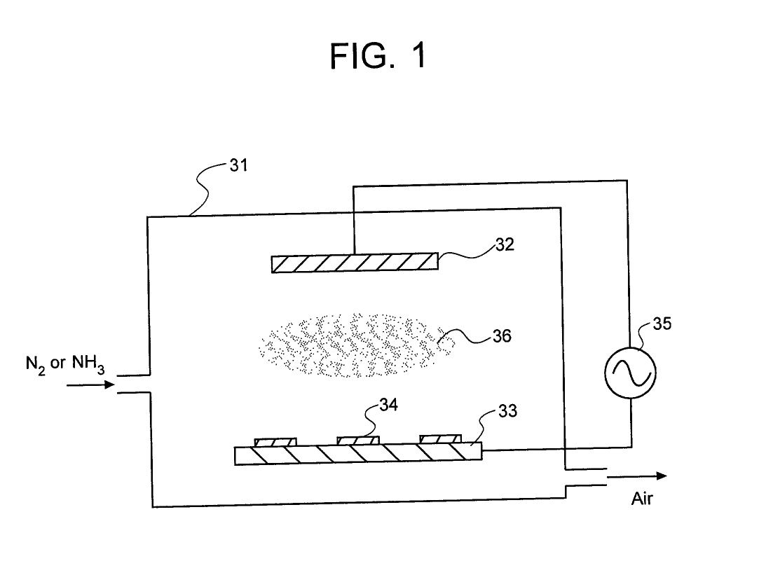

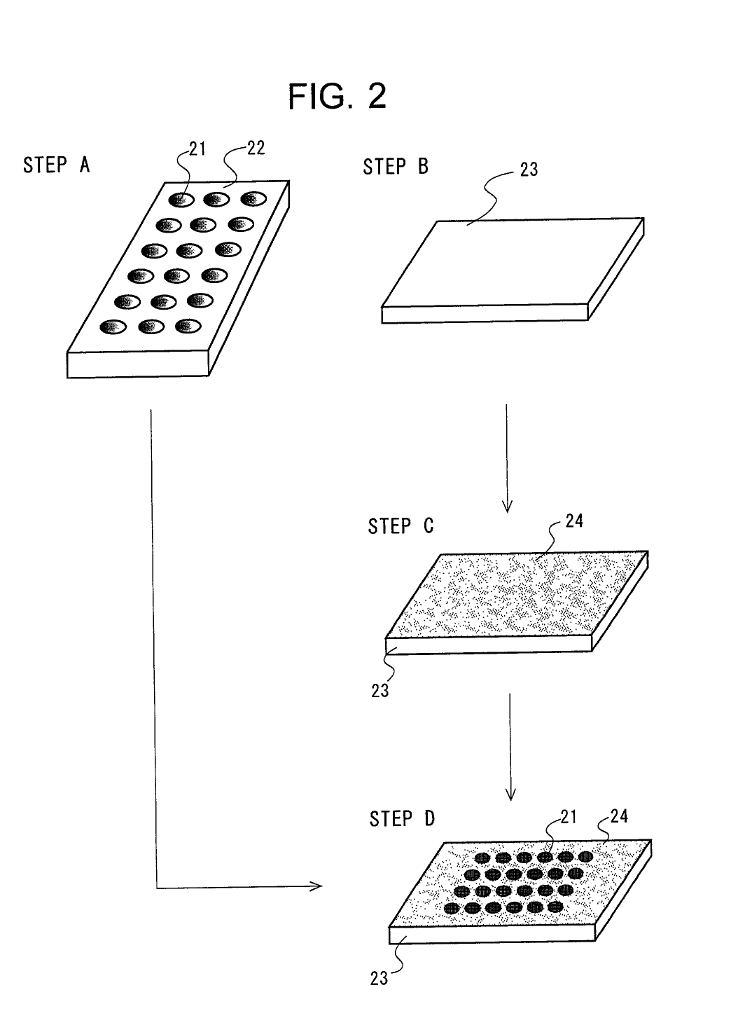

By using the apparatus as shown in FIG. 1, a glass substrate 34 was placed on a substrate 33 in a vacuum chamber 31, an amino-modified DLN film was deposited to a film thickness of 0.3 μm on the glass substrate 34. The vacuum chamber was evacuated to maintain the predetermined degree of vacuum, and the source gas was introduced in the chamber. RF power was applied with self-bias between the substrate 33 and a counter electrodes 32 from an AC power source 35 to form a plasma 36. The conditions used were:

silicone oil: dimethylsilicone oil manufactured by Shinetsu Chemical under the product name of KF96SS,

temperature: 350° C.,

degree of vacuum in the chamber: 10⁻⁴ Torr,

gas source: N₂,

source gas flow rate: 5 SCCM, and

power applied: RF, 500 W.

After completing the chemical processing required for the amide bonding, the DNA chip was produced by the steps as shown in FIG. 2, steps A to D. To be more specific, the probe DNA in the microplate 22 was attached to the pin, and this probe DNA on the pin was brought in contact with the glass plate 23 having the polymer film formed thereon for spotting. This procedure was repeated until all probe DNAs in the microplate 22 had been spotted to thereby produce the DNA chip as shown in FIG. 2, step D.

The hybridization of the DNA chip was accomplished by placing both the DNA chip having the probe DNAs bonded to the glass plate and the sample DNA that had been labeled with a fluorescent substance in a hybridization solution for hybridization. The hybridization solution was a mixed solution comprising formaldehyde, SSC (NaCl, trisodiumcitrate), SDS (sodium dodecyl sulfate), EDTA (ethylenediaminetetraacetic acid), distilled water, and the like, and the mixing ratio was varied depending on the nature of the DNA used.

Next, the sample DNA having the fluorescent label remaining on the glass plate was washed in a water tank or the like to thereby remove the sample DNA which failed to bind to the probe DNA.

In this process, almost all of the probe DNA that had been bonded to the substrate remained on the substrate without becoming peeled off, and it was confirmed that the DNA is not peeled off the DNA chip in the washing procedure.

Hybridization was then detected by exciting the fluorescent label on the sample DNA which became bonded to the probe DNA with the light energy from the predetermined light source, and detecting the light emitted by the excitation of the fluorescent label with a photosensor such as CCD.

It was then confirmed that the desired hybridization had been adequately accomplished. It was also confirmed that, in this process, almost all of the probe DNA that had been bonded to the substrate remained on the substrate without becoming peeled off, and the DNA would not become peeled off the DNA chip in the washing procedure with water.

It was also indicated that the method of the present invention wherein the step of amination is unnecessary is quite effective in reducing the production cost since the number of production steps has been reduced in the present invention at least by two steps.

### Example 2

The procedure of Example 1 was repeated except that the amino-modified DLN film was formed by using an amino-modified silicone oil as the silicone oil and no gas was introduced from the gas source. The resulting product was evaluated for its performance as a DNA chip. It was then confirmed that the performance of this DNA chip was substantially equivalent to the DNA chip of Example 1.

### Example 3

The procedure of Example 1 was repeated except that the film formed was not the DLN film but a DLC film. The conditions used were:

gas source: ethylene

source gas flow rate: 1 to 4 SCCM,

gas source: N₂,

source gas flow rate: 5 SCCM, and

degree of vacuum in the chamber: 10⁻⁴ Torr,

power applied: RF, 500 W.

The DNA chip was produced by repeating the procedure of Example 1 except for the conditions as described above, and it was then confirmed that the performance of the resulting DNA chip was substantially equivalent to that of Example 1 while regulation of source gases was necessary. However, it is to be noted that the DLN film was superior in adhesion to the underlying substrate, stability, heat resistance, light resistance, and mechanical strength compared to the DLC film, indicating that the DLN film had the quality which could endure even higher number of repetitive use compared to the DLC film.

### MERITS OF THE INVENTION

As described above, this invention provides a method for producing a DNA chip which can be accomplished in simple steps at a low cost, and wherein use of the resulting DNA chip reduces loss of probes and sample substances in the washing step enabling efficient use of such probe and sample. Also provided is a DNA chip produced by such method.

Japanese Patent Application No. 160935/2001 is incorporated herein by reference.

Although some preferred embodiments have been described, many modifications and variations may be made thereto in the light of the above teachings. It is therefore to be understood that, within the scope of the appended claims, the invention may be practiced otherwise than as specifically described.

1. A method for producing a DNA chip comprising a substrate and a DNA-binding layer formed on the substrate wherein said DNA-binding layer is a diamond like film having a DNA-binding group; comprising the steps of
reducing pressure of a vacuum chamber to a predetermined degree of vacuum;
feeding the chamber with a gas which is the source of said diamond like film;
feeding the chamber with a gas which is the source of nitrogen; and
forming the diamond like film having a DNA-binding group on the substrate by CVD.

2. The method for producing a DNA chip according to claim 1 wherein said diamond like film is a diamond like nanocomposite (DLN) film comprising at least carbon, silicon, oxygen, and hydrogen.

3. The method for producing a DNA chip according to claim 1 wherein said diamond like film is a diamond like carbon (DLC) film comprising at least carbon and hydrogen.

4. The method for producing a DNA chip according to claim 1 wherein the source gas for said diamond like film is the one obtained by heating a silicone oil.

5. The method for producing a DNA chip according to claim 4 wherein said nitrogen source is included in the silicone oil.

6. A DNA chip which is produced by the method of claim 1, and which has a DNA-binding layer having a DNA-binding group.
